(19) United States
(12) Patent Application Publication
(10) Pub. No.: US 2013/0190621 A1
(43) Pub. Date: Jul. 25, 2013

(54) THROMBOSIS WARNING SYSTEM

(71) Applicant: John Somberg, Lake Forest, IL (US)

(72) Inventor: John Somberg, Lake Forest, IL (US)

(73) Assignee: ACADEMIC PHARMACEUTICALS INCORPORATED, Lake Bluff, IL (US)

(21) Appl. No.: 13/560,695

(22) Filed: Jul. 27, 2012

Related U.S. Application Data
(60) Provisional application No. 61/512,905, filed on Jul. 28, 2011.

(51) Int. Cl. A61B 5/02

(52) U.S. Cl. CPC A61B 5/02007
USPC 600/438; 600/481; 600/479; 604/66

(57) ABSTRACT

The present invention relates to a device for detecting the potential formation of a thrombus in a patient on a ventricular assist device. The device uses an energy source and sensor to detect changes in rheology of the patient.

### CROSS-REFERENCE TO RELATED APPLICATIONS

The present application claims priority benefit under 35 U.S.C. §119(e) of U.S. Provisional Patent Application Ser. No. 61/512,905 filed 28 Jul. 2011. The disclosure this application is incorporated herein by reference.

### BACKGROUND OF THE INVENTION

Patients with severe heart failure are often placed on extracorporial circulating pumps to augment their own cardiac function either to support cardiac circulation for a period of time or as a “bridge” to cardiac transplant. This procedure may be done for both children and adults. Children, due to their small size, are especially difficult to place on the cardiac pump system and are especially prone to thrombosis formation probably due to the very small size of the pumps. The thrombosis is often responsible for embolization and can cause neurologic events. Neurologic events can be devastating and a serious cause of morbidity and mortality in patients.

To prevent the occurrence of the thrombotic events and neurologic morbidity and mortality, anti-coagulant and anti-platelet agents are often administered to patients. Despite this often aggressive therapy, thrombotic events are still frequent and problematic. Some devices use filters to attempt to reduce thrombotic events, but the filters themselves can be a nidus for thrombosis formation. Some systems have a transparent chamber such as the Berlin Heart Pediatric VAD that physicians, nurses or other trained personnel can observe to see if thrombus forms and can change the chamber components to remove thrombosis and/or clot. Still embolization may occur despite these precautions because the thrombus is not caught in time by visual inspection.

It is therefore desirable to develop improved ventricular assist devices that are capable of alerting a technician of the formation or potential formation of a thrombus.

### SUMMARY OF THE INVENTION

The present invention relates to an energy source and sensor operably connected to a ventricular assist device.

The present invention also relates to an energy source, sensor, and an alarm system operably connected to a ventricular assist device, the alarm system being capable of alerting medical personnel of possible changes in blood clotting.

The present invention also relates to a ventricular assist device to which is operably connected an energy system and sensor.

The present invention also relates to a ventricular assist device to which is operably connected an energy system, sensor, and an alarm system capable of alerting medical personnel of possible changes in blood clotting.

These and other objects, which will become apparent during the following detailed description, have been achieved by the inventors' discovery of an energy source and sensor that can be operably connected to a ventricular assist device.

### BRIEF DESCRIPTION OF THE DRAWINGS

FIG. 1: A ventricular assist device (VAD) 100 is shown having two entry and two exit conduits (e.g., cannulas) 102. The VAD has a transparent chamber 101. There is an energy source (e.g., light or ultrasound) 104 on one side of the transparent chamber and a sensor 103 on a side opposite the energy source and capable of detecting changes in the energy source.

FIG. 2: A ventricular assist device (VAD) 200 is shown having two entry and two exit conduits (e.g., cannulas) 202, at least one of which is transparent. The VAD has an opaque chamber 201. There is an energy source (e.g., light or ultrasound) 204 on one side of the transparent conduit and a sensor 203 on a side opposite the energy source and capable of detecting changes in the energy source.

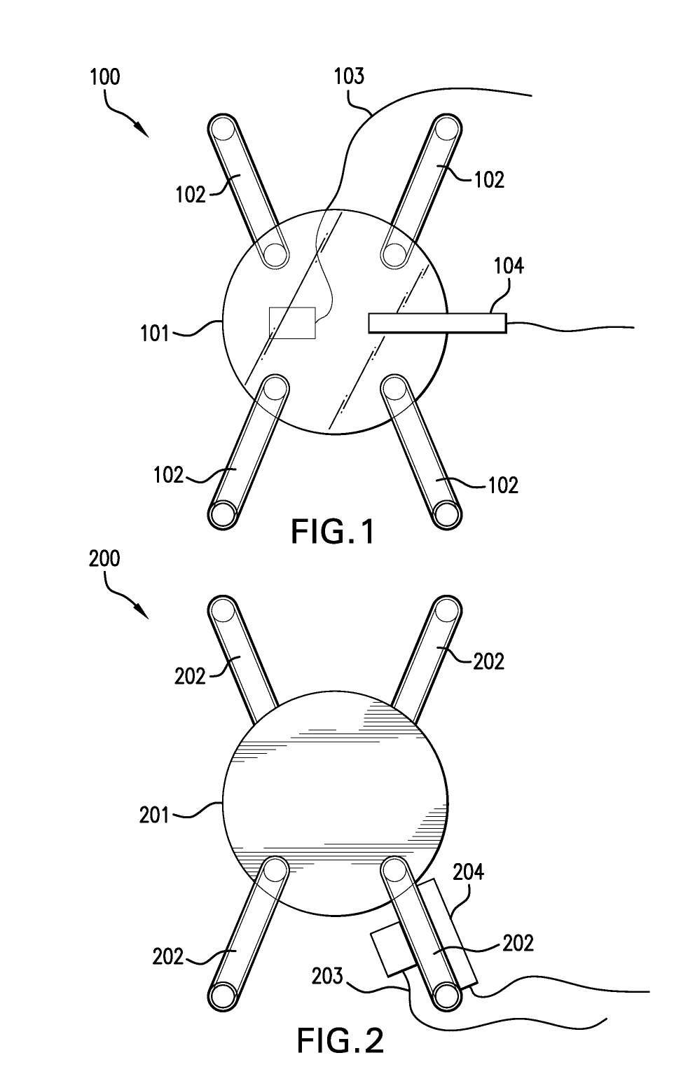

### DETAILED DESCRIPTION OF THE INVENTION

The present invention relates to a VAD, which includes extracorporial assist devices (EAD, to which is operably connected an energy source and a sensor. The sensor being capable of detecting changes in energy from the energy source. Such changes would be indicative of when a thrombus forms, begins to form, or there is a change in the patient's blood rheology that increases the propensity of the blood to develop a thrombus. An alarm system can be operably connected to the sensor. The alarm system can be capable of alerting medical personnel of possible changes in blood clotting that could lead to embolization and/or stroke in patients with cardiac dysfunction on the VAD. The present invention is designed to detect the formation of thrombus in a VAD that alerts medical personnel (e.g. via electronic communication (e.g., text, telephone, or e-mail message system), optical communication (e.g., activation of a warning light), aural communication (e.g., activation of an alarm sound), or a combination thereof), so appropriate action can be taken.

It can be useful for the device to be connected to a computing device (e.g., a desktop, laptop, and/or handheld device that is optionally electronically connected to a network (e.g., via hardwire or wirelessly) that uses a change in electrical conductance (or field or impedance), light transmittance, or ultrasound transmittance, that can detect the tendency for blood thrombus to form and can, when an adjustable threshold is met, activate the automatic administration of a heparin solution and/or anti-platelet agents to be administered to a patient's circulation to reduce a thrombotic tendency.

The present device is designed to catch the thrombus formation early on, when it just occurs, to reduce the potential for the thrombus embolize.

In an aspect, the present invention provides a device comprising:

(a) an energy source that transmits through a portion of a VAD, and

(b) a sensor in association with the energy source and capable of sensing changes in energy from the energy source.

In another aspect,

i. the energy source is positioned to send energy through a patient's blood and to the sensor;

ii. the energy changes when a thrombus forms, begins to form, or there is a change in the patient's blood rheology that increases the propensity of the blood to develop a thrombus;

iii. and, the sensor is capable of detecting the energy change event.

In another aspect, the energy source is light source that transmits light through a transparent chamber in the VAD to a light-sensitive sensor. As thrombi forms or the blood becomes more likely to thrombose, the percent light transmittance changes. Besides light, other sources of energy may be employed to detect thrombosis. For example, ultrasound energy may be transmitted through a pump chamber or tubing either from or to the patient. A low voltage electric current may also be used. The change in conduction (or field or impedance) in blood may be correlated with a propensity for thrombus formation and thus the potential for embolization.

The VAD to which the present invention is operably connected will typically have a transparent chamber and/or conduit when light energy is used in the device of the present invention. It may also have a translucent or opaque chamber, though typically a different energy source (e.g., ultrasound or electricity) will be used. The present invention may comprise more than one energy source and more than one sensor. When more than one energy source and sensor are present, they may be positioned on the chamber, conduit, or both of the VAD.

In another aspect, the device, further comprises: an alarm system that is activated by the sensor when an event occurs. The alarm is intended to warn of a thrombotic problem. The alarm can be any appropriate signal to notify a responsible party, for example, an aural message (e.g., activation of an audio signal such as an alarm sound), an optical message (e.g., activation of a warning light such as a flashing light(s) on a display), and/or an electronic message (e.g., text, telephone, or e-mail message) sent to a responsible party (e.g., physician, nurse, or attendant).

In another aspect, the energy source and sensor of the present invention can be retrofitted to a VAD. Alternatively, the energy source and sensor can be incorporated into the construction of a VAD (e.g., the device will be integrated into the VAD).

In another aspect, the device, further comprises: a VAD.

In another aspect, the VAD, comprises at least one chamber, at least two entry conduits (e.g., cannulas), and at least two exit conduits (e.g., cannulas). In another aspect, at least one chamber of the VAD is transparent. In another aspect, at least one conduit of the VAD is transparent.

In another aspect, a computing device is electronically connected to the sensor.

In another aspect, the device further comprises: an automated drug delivery system capable of administering a drug to the patient and electronically connected to the computing device. When the computer is notified of a change in energy transmittance by the sensor, it is capable of activating the drug delivery system and thereby causing administration of an agent suitable to reduce a thrombotic tendency in the patient or to dissolve a formed or partially formed thrombus. Examples of agents include heparin, ticagrelor, bivalirudin, SK (streptokinase), and rtPA (recombinant tissue plasminogen activator)

In additional aspects, the energy source and sensor of the present invention can, independently, be located inside of the VAD (e.g., attached to an interior surface of a chamber or conduit) or on the outside of the VAD (e.g., attached to an exterior surface). The energy source and sensor can, independently, be directly attached to an interior or exterior surface (e.g., surface mounted) or be located adjacent to one of these surfaces.

Numerous modifications and variations of the present invention are possible in light of the above teachings. It is therefore to be understood that within the scope of the appended claims, the invention may be practiced otherwise that as specifically described herein.

What is claimed is:

1. A device that would alert medical personnel to the development of thrombus or a change in blood rheology to be more thrombogenic, the device, comprising:
a. an energy source that transmits through a portion of a ventricular assist device (VAD), and
b. a sensor in association with the energy source and capable of sensing changes in energy from the energy source.

2. The device of claim 1, wherein:
i. the energy source is positioned to send energy through a patient's blood and to the sensor,
ii. the energy changes when a thrombus forms, begins to form, or there is a change in the patient's blood rheology that increases the propensity of the blood to develop a thrombus; and,
iii. the sensor is capable of detecting the energy change event.

3. The device of claim 2, wherein the energy is light.

4. The device of claim 2, wherein the energy is ultrasound.

5. The device of claim 2, wherein the energy is electricity and the sensor is capable of detecting changes in conductance.

6. The device of claim 2, wherein the energy is electricity and the sensor is capable of detecting changes in impedance.

7. The device of claim 2, wherein the energy is electricity and the sensor is capable of detecting changes in electrical field.

8. The device of claim 2, further comprising: an alarm system that is activated by the sensor when an event occurs.

9. The device of claim 8, wherein the alarm transmits a signal to a medical personnel when an event occurs, the signal being selected from an audio signal, a light signal, an electronic signal, and a combination thereof.

10. The device of claim 9, wherein the signal is an electronic signal.

11. The device of claim 10, wherein the electronic signal is selected from: an e-mail message, a text message, a voice message, or a combination thereof.

12. The device of claim 2, further comprising a VAD.

13. The device of claim 12, wherein the VAD, comprises at least one chamber, at least two entry conduits, and at least two exit conduits.

14. The device of claim 13, wherein the chamber is transparent.

15. The device of claim 13, wherein at least one of the conduits is transparent.

16. The device of claim 2, further comprising: a computing device electronically connected to the sensor.

17. The device of claim 16, further comprising: an automated drug delivery system capable of administering a drug to the patient and electronically connected to the computer.

18. The device of claim 17, wherein the computing device, when notified of a change in energy transmittance by the sensor, is capable of activating the drug delivery system and thereby causing administration of an agent suitable to reduce a thrombotic tendency in the patient.

19. The device of claim 10, wherein the energy source and sensor are located inside of the VAD.

20. The device of claim 10, wherein the energy source and sensor are located on the outside of the VAD.
